(19) United States
(12) Patent Application Publication
(10) Pub. No.: US 2016/0193046 A1
(43) Pub. Date: Jul. 7, 2016

(54) VALVULOPLASTY BALLOON AND VALVE STENT DEPLOYMENT CATHETER

(71) Applicant: Romeo Majano, Coral Gables, FL (US)

(72) Inventor: Romeo Majano, Coral Gables, FL (US)

(21) Appl. No.: 15/045,833

(22) Filed: Feb. 17, 2016

Related U.S. Application Data
(63) Continuation-in-part of application No. 14/226,861, filed on Mar. 27, 2014.

(51) Int. Cl. A61F 2/24

(52) U.S. Cl. CPC A61F 2/2427; A61F 2/2433; A61F 2/2436; A61F 2002/484

(57) ABSTRACT

A heart valvuloplasty and stent valve deployment catheter assembly includes a single unitary catheter body having a proximal end and a distal end. The catheter body has a lumen extending between the proximal end and the distal end. A guide wire extends through the lumen proximally of the proximal end and distally of the distal end. A valvuloplasty balloon is disposed on the body. The balloon is located proximate to the distal end of the body. At least one radiopaque marker is disposed on the balloon. A valvuloplasty balloon inflation connection is located distally of the proximal end of the catheter body. A valvuloplasty inflation lumen extends partially through the catheter body and provides fluid communication between the valvuloplasty balloon inflation connection and the balloon. A stent valve is disposed over the body. The stent valve can be self-expanding or balloon expanding.

### CROSS-REFERENCE TO RELATED APPLICATION

The present application is a Continuation-in-Part application of U.S. patent application Ser. No. 14/226,861, filed on Mar. 27, 2014, which is incorporated herein by reference in its entirety.

### FIELD OF THE INVENTION

The present invention relates to a unitary catheter assembly that is used to predilate a calcified or otherwise damaged heart valve and to deploy a stent valve across the valve to replace the valve.

### BACKGROUND OF THE INVENTION

Aortic stenosis is a common progressive valvular heart disease that produces obstruction to left ventricular outflow. This results in pressure hypertrophy of the left ventricle, and the symptoms of dyspnea, angina, syncope and finally sudden death. The traditional natural history of this disease has led to surgical Aortic Valve Replacement techniques as the treatment. Nevertheless, in recent years, non-surgical or hybrid approaches to Transcatheter Aortic Valve Replacement (TAVR) have evolved and have received FDA approval. The typical approaches are trans-femoral (TF) versus Trans-apical (via the apex of the heart—hence the term hybrid procedure) procedures. The current biological valves implanted are mounted on a balloon expandable or self expandable stent that is inflated at the aortic valve to “replace” the diseased valve.

This technique first requires introduction of a guide wire thought the stenosed aortic valve (AV) (either from the aortic side (TF) approach or via the apex of the heart (TA) approach). This is followed by the insertion of a valvuloplasty catheter, which is inflated and then deflated at the AV site to make way for a second, valve delivery catheter, which is a bulkier transcatheter aortic replacement delivery catheter with a balloon mounted or self-expanding stent. When at the implant site, the valve mounted stent is deployed and expanded. The valve delivery catheter is then removed, leaving the stent valve, which replaces the damaged heart valve.

This procedure is analogous to traditional coronary or peripheral vascular angioplasty and stent placement. In these procedures a wire is introduced through the blockage followed by a predilatation balloon, which in inflated, deflated and then removed. Following this, a separate stent delivery balloon expandable or self-expandable stent catheter is advanced to deliver the stent at site.

An improved device for performing the above procedure without requiring the insertion and removal of two separate catheters is required.

### BRIEF SUMMARY OF THE INVENTION

Briefly, the present invention provides a heart valvuloplasty and stent valve deployment catheter assembly having a single unitary catheter body having a proximal end and a distal end. The catheter body has a lumen extending between the proximal end and the distal end. A guide wire extends through the lumen proximally of the proximal end and distally of the distal end. A valvuloplasty balloon is disposed on the body. The valvuloplasty balloon is located proximate to the distal end of the body. At least one radiopaque marker is disposed on the valvuloplasty balloon. A valvuloplasty balloon inflation connection is located distally of the proximal end of the catheter body. A valvuloplasty inflation lumen extends partially through the catheter body and provides fluid communication between the valvuloplasty balloon inflation connection and the valvuloplasty balloon. A stent valve is disposed over the body a distance of about 5 millimeters proximally of the valvuloplasty balloon. A stent valve sheath is slidingly disposed over a portion of the catheter body and the stent valve.

Further, the present invention provides a heart valvuloplasty and stent valve deployment catheter assembly comprising a single unitary catheter body having a proximal end, a distal end, a guide wire lumen extending between the proximal end and the distal end, and a valvuloplasty balloon inflation lumen extending at least partially through the catheter body. A valvuloplasty balloon is located proximate to the distal end and in fluid communication with the valvuloplasty balloon inflation lumen. A stent valve is disposed on the body, proximally of the valvuloplasty balloon.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side elevational view of a sheathless catheter assembly according to a first embodiment of the present invention;

FIG. 2 is a sectional view of the sheathless catheter assembly of FIG. 1, taken along lines 2-2 of FIG. 1;

FIG. 3 is a sectional view of the sheathless catheter assembly of FIG. 1, taken along lines 3-3 of FIG. 1;

FIG. 4 is a sectional view of the sheathless catheter assembly of FIG. 1, taken along lines 4-4 of FIG. 1;

FIG. 5 is a sectional view of the sheathless catheter assembly of FIG. 1, taken along lines 5-5 of FIG. 1;

FIG. 6 is a flow chart illustration an exemplary operation of the sheathless catheter assembly of FIG. 1;

FIG. 7 is a side elevational view, in section, of a coronary artery showing a guide wire being passed through a blockage in the artery;

FIG. 8 is a side elevational view, in section, of the coronary artery of FIG. 8, with a predilatation balloon of the sheathless catheter assembly of FIG. 1 inflated at the site of the blockage;

FIG. 9 is a side elevational view, in section, of the coronary artery of FIG. 8, with a stent inflation balloon of the sheathless catheter assembly of FIG. 1 inflated to expand a stent at the site of the blockage;

FIG. 10 is a side elevational view of a valvuloplasty catheter assembly with a self-expanding stent valve according to a second embodiment of the present invention;

FIG. 11 is a sectional view of the valvuloplasty catheter assembly of FIG. 10, taken along lines 11-11 of FIG. 10;

FIG. 12 is a sectional view of the valvuloplasty catheter assembly of FIG. 10, taken along lines 12-12 of FIG. 10;

FIG. 13 is a sectional view of the valvuloplasty catheter assembly of FIG. 10, taken along lines 13-13 of FIG. 10;

FIG. 14 is a front elevational view of a valve used with the catheter assembly of FIG. 10 after valve deployment;

FIG. 15 is a side elevational view of the valvuloplasty catheter assembly shown in FIG. 10, with a dilatation balloon expanded across a heart valve;

FIG. 16 is a side elevational view of the valvuloplasty catheter assembly shown in FIG. 10, with a sheathed valve located across the heart valve;

FIG. 17 is an enlarged elevational view of the valvuloplasty catheter assembly shown in FIG. 10, with the sheath retracted and the valve expanded across the heart valve; and

FIG. 18 is a side elevational view of a valvuloplasty catheter assembly with a balloon expandable stent valve according to a third embodiment of the present invention.

### DETAILED DESCRIPTION OF THE INVENTION

In the drawings, like numerals indicate like elements throughout. Certain terminology is used herein for convenience only and is not to be taken as a limitation on the present invention. The terminology includes the words specifically mentioned, derivatives thereof and words of similar import. As used herein, the term “fluid” can mean and material that flows, including a liquid or a gas. The term “proximal” defines a location closer to the inserting physician and the term “distal” defines a location farther from the inserting physician. The term “about” is interpreted to mean a range of ±10% of the listed value.

The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

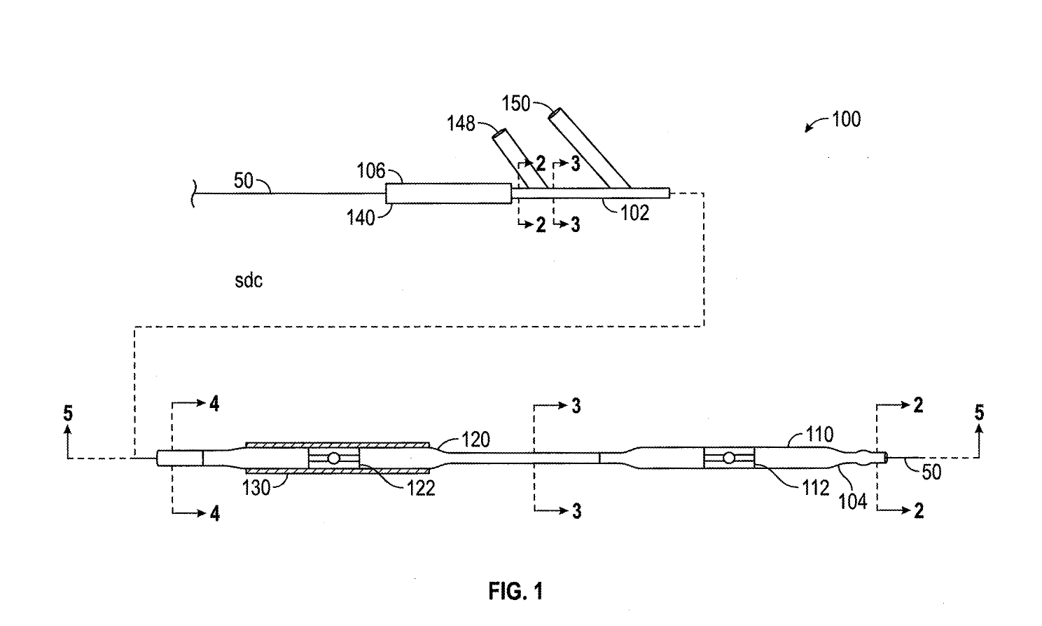

Referring to FIGS. 1-5, a first exemplary embodiment of a catheter assembly 100 according to the present invention is shown. Catheter assembly 100 is used to open up blockages within coronary arteries.

Catheter assembly 100 is specifically designed for use within narrow coronary arteries that have an inside diameter of typically 6 French or less. The fact that catheter assembly 100 is sheathless allows catheter assembly 100 to be inserted into such narrow arteries. Sheathed catheters are too wide in diameter to fit into these arteries, given the additional width of the sheath itself.

Catheter assembly 100 has a unitary catheter body 102 that incorporates a predilatation balloon 110 at a distal end 104 of body 102 and a combination stent balloon 120 and stent 130 are located proximally of predilatation balloon 110. In an exemplary embodiment, predilatation balloon 110 has a deflated diameter of about 2.5 millimeters and a length of about 15 millimeters. Also, stent balloon 110 can include a radiopaque marker 112 disposed on an exterior thereof to allow for imaging and locating stent balloon 110 within a blood vessel 52 (shown in FIG. 8) during an angioplasty procedure.

In an exemplary embodiment, stent balloon 120 and stent 130 are located between about 10 millimeters and about 15 millimeters proximally from predilatation balloon 110. In an exemplary embodiment, catheter body 102 can be constructed from polytetrafluoroethylene, although those skilled in the art will recognize that catheter body 102 can be constructed from other material. Further, each of predilatation balloon 110 and stent balloon 120 inflate upon introduction of an inflation fluid therein, and contract toward their original size upon release or withdrawal of the inflation fluid from inside each of predilatation balloon 110 and stent balloon 120.

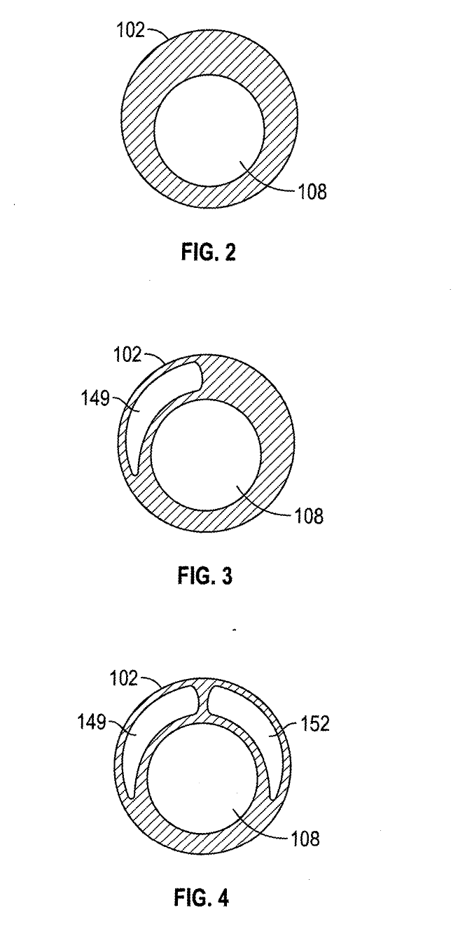

Catheter assembly 100 also includes a proximal end 106. As shown in FIG. 2, catheter assembly 100 includes a guide wire lumen 108 that extends from proximal end 106, through catheter body 102, to distal end 104. Guide wire lumen 108 is sized to allow a guide wire 50 to extend fully therethrough between proximal end 102 and distal end 104.

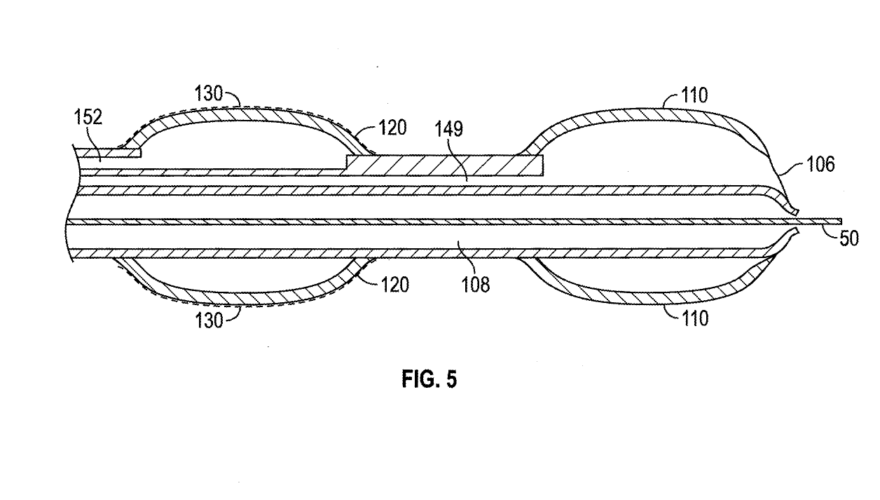

A predilatation balloon inflation connection 148 is located distally of proximal end 106. Predilatation balloon inflation connection 148 is releasably connectable to an inflation source (not shown) that provides an inflation fluid such as, for example, saline, to inflate predilatation balloon 110. As shown in FIGS. 3 and 5, a predilatation inflation lumen 149 provides fluid communication between predilatation balloon inflation connection 148 and predilatation balloon 110. Predilatation inflation lumen 149 extends through stent balloon 120.

A stent balloon inflation connection 150 is located distally of predilatation balloon inflation connection 148. While stent balloon inflation connection 150 is shown as being located distally of predilatation balloon inflation connection 148, those skilled in the art will recognize that stent balloon inflation connection 150 can be located proximally of predilatation balloon inflation connection 148 without departing from the scope of the present invention.

Stent balloon inflation connection 150 is releasably connectable to an inflation source (not shown) that provides an inflation fluid such as, for example, saline, to inflate stent balloon 120. The same fluid source that is used to inflate predilatation balloon 110 can be used to inflate stent balloon 120. As shown in FIGS. 4 and 5, a stent balloon inflation lumen 152 provides fluid communication between stent balloon inflation connection 150 and stent balloon 120. Referring back to FIG. 1, stent balloon 120 includes at least one radiopaque marker 122 that allows the treating physician to locate stent balloon 120 within blood vessel 52.

Stent 130 is an expandable stent as is well known in the art. Stent 130 is not self-expanding, but is expanded by the inflation of stent balloon 120. Stent 130 remains expanded after stent balloon 120 is deflated. Further, in an exemplary embodiment, stent 130 has an expanded size of customarily known, industry standard, and well-used coronary stents within typical ranges of between about 2.5 millimeters and about 4 millimeters in diameter and between about 12 millimeters and about 33 millimeters in length. Additionally, in an exemplary embodiment, stent 130 does not include a graft, although those skilled in the art will recognize that a graft may be utilized with stent 130.

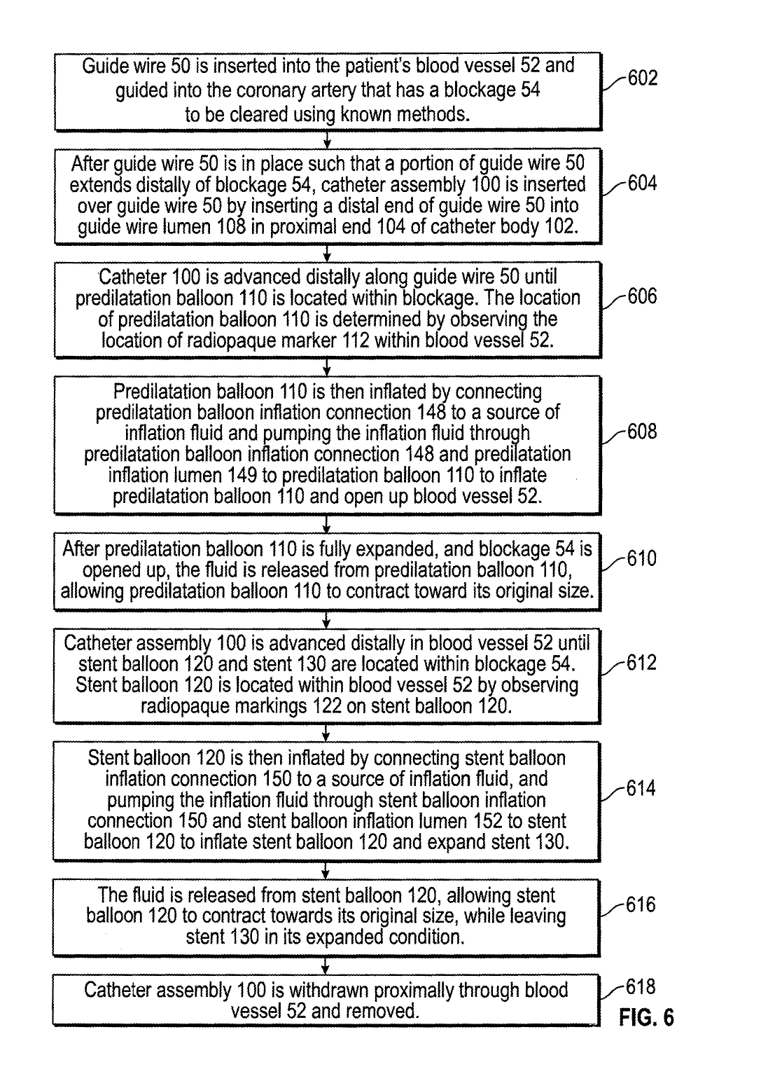

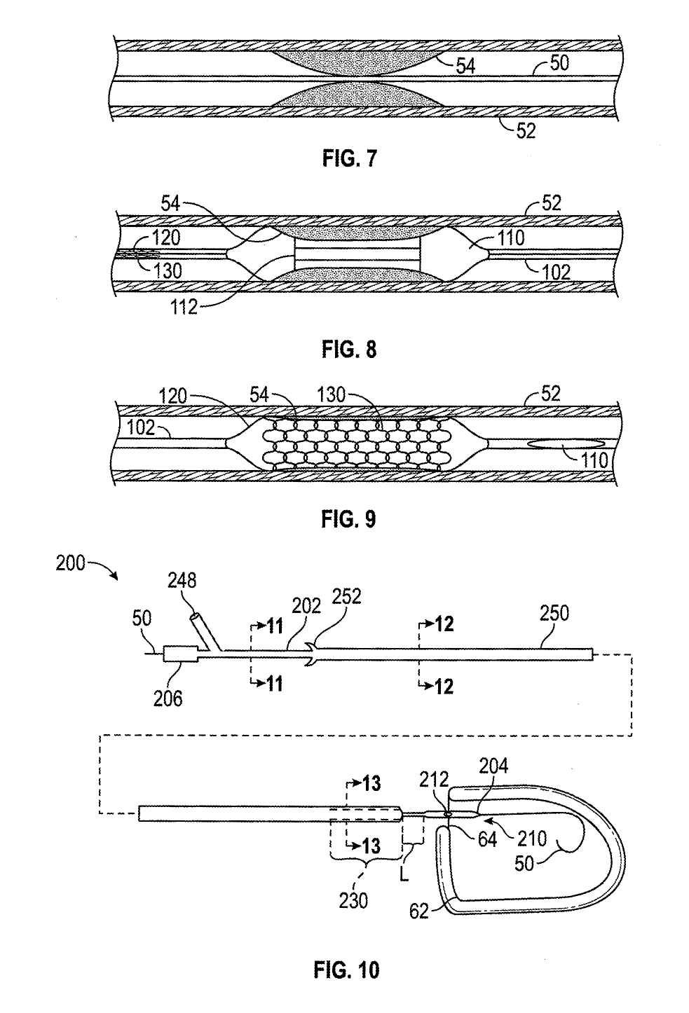

To use catheter assembly 100, and as explained in flowchart 600 of FIG. 6, in step 602, guide wire 50 is inserted into the patient's blood vessel 52, such as, for example, through a femoral artery, and guided into the coronary artery that has a blockage 54 to be cleared using known methods, as shown in FIG. 7. After guide wire 50 is in place such that a portion of guide wire 50 extends distally of blockage 54, in step 604, catheter assembly 100 is inserted over guide wire 50 by inserting a distal end of guide wire 50 into guide wire lumen 108 in proximal end 104 of catheter body 102.

In step 606, catheter 100 is advanced distally along guide wire 50 until predilatation balloon 110 is located within blockage 54, as shown in FIG. 8. The location of predilatation balloon 110 is determined by observing the location of radiopaque marker 112 within blood vessel 52 using known techniques. Predilatation balloon 110 is then inflated in step 608 by connecting predilatation balloon inflation connection 148 to a source of inflation fluid (not shown), and pumping the inflation fluid through predilatation balloon inflation connection 148 and predilatation inflation lumen 149 to predilatation balloon 110 to inflate predilatation balloon 110 and open up blood vessel 52, as shown in FIG. 8.

After predilatation balloon 110 is fully expanded, and blockage 54 is opened up, in step 610, the fluid is released from predilatation balloon 110, allowing predilatation balloon 110 to contract toward its original size. In step 612, catheter assembly 100 is advanced distally in blood vessel 52 until stent balloon 120 and stent 130 are located within blockage 54. Stent balloon 120 is located within blood vessel 52 by observing radiopaque markings 122 on stent balloon 120.

In step 614, stent balloon 120 is then inflated by connecting stent balloon inflation connection 150 to a source of inflation fluid (not shown), and pumping the inflation fluid through stent balloon inflation connection 150 and stent balloon inflation lumen 152 to stent balloon 120 to inflate stent balloon 120 and expand stent 130, as shown in FIG. 9. In step 616, the fluid is released from stent balloon 120, allowing stent balloon 120 to contract toward its original size, while leaving stent 130 in its expanded condition. In step 618, catheter assembly 100 is withdrawn proximally through blood vessel 52 and removed.

The inventive catheter assembly and method of the present invention obviates the need for two or more catheters, along with several catheter exchanges or manipulations to perform the method. This in turn decreases the chance of losing the position of the guide wire during the catheter balloon extraction. Further, increased pushability and turgor of the inventive assembly may improve the ease of advancing the catheter through calcific and tortuous arteries, especially when part of the inventive catheter assembly is already distally past the blockage.

Additionally, the lower cost of a single catheter, along with less time and radiation exposure required for catheter laboratory (Cath Lab) personnel may significantly decrease the cost of an angioplasty procedure. Further, patient safety and convenience may be enhanced by eliminating exchanges of catheters over the guide wire.

An alternative embodiment of a catheter assembly 200 according to the present invention is shown in FIGS. 10-16. Catheter assembly 200 is used to install a stent valve as a replacement for a damaged or calcified coronary valve. Catheter assembly 200 incorporates a valvuloplasty balloon and a self-expanding stent valve in the same assembly, eliminating the need for separate insertions of a valvuloplasty balloon and a stent valve by separate catheters or other insertion devices. Catheter assembly 200 reduces the amount of catheters required to perform a stent valve insertion procedure, reducing the risk of injury to the patient.

Catheter assembly 200 has a unitary catheter body 202 that incorporates a valvuloplasty balloon 210 at a distal end 204 of body 202 and a self-expanding stent valve 230 located proximally of valvuloplasty balloon 210. In an exemplary embodiment, valvuloplasty balloon 210 has a deflated diameter of between about 18 millimeters and about 25 millimeters and a length of about 5 centimeters. Also, valvuloplasty balloon 210 can include a radiopaque marker 212 disposed on an exterior thereof to allow for imaging and locating valvuloplasty balloon 210 within a chamber of a heart 62, such as a left ventricle, during a radiographic or fluoroscopic procedure.

In an exemplary embodiment, stent valve 230 is located a length “L” of about 5 millimeters proximally from valvuloplasty balloon 210. In an exemplary embodiment, the distance between distal end 204 and stent valve 230 is minimized because catheter assembly 200 is at least partially inserted into a patient's heart to deploy stent valve 230 across a heart valve 64, leaving little room for distal end 204 in the heart 62.

In an exemplary embodiment, catheter body 202 can be constructed from polytetrafluoroethylene, although those skilled in the art will recognize that catheter body 202 can be constructed from other material. Further, valvuloplasty balloon 210 inflates upon introduction of an inflation fluid therein, and contract toward its original size upon release or withdrawal of the inflation fluid from inside of valvuloplasty balloon 210.

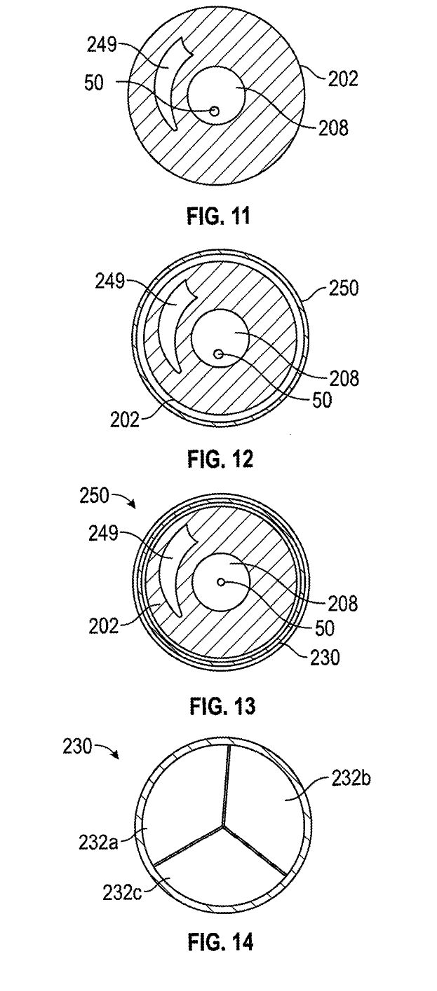

Catheter assembly 200 also includes a proximal end 206. As shown in FIG. 11, catheter assembly 200 includes a guide wire lumen 208 that extends from proximal end 206, through catheter body 202, to distal end 204. Guide wire lumen 208 is sized to allow a guide wire 50 to extend fully therethrough between proximal end 202 and distal end 204.

A valvuloplasty balloon inflation connection 248 is located distally of proximal end 206. Valvuloplasty balloon inflation connection 248 is releasably connectable to an inflation source (not shown) that provides an inflation fluid such as, for example, saline, to inflate valvuloplasty balloon 210. As shown in FIGS. 11-13, a valvuloplasty inflation lumen 249 extends partially through catheter body 202 and provides fluid communication between valvuloplasty balloon inflation connection 248 and valvuloplasty balloon 210.

In an exemplary embodiment, as shown in FIG. 14, stent valve 230 is a tricuspid one-way valve having valve flaps 232a, 232b, 232c that are operable between a closed position in which blood flow is restricted from passing through stent valve 230, and an open position in which blood flow is allowed to pass through stent valve 230.

Referring back to FIGS. 10, 12, and 13, stent sheath 250 is disposed at least partially over catheter body 202, including over stent valve 230. Stent sheath 250 extends proximally toward predilatation balloon inflation connection 248, and can optionally include a handle 252 that allow stent sheath 250 to be slid proximally along catheter body 202 to release stent valve 230. In the event that stent valve 230 needs to be repositioned while stent valve 230 is still disposed over catheter body 202, sheath 250 can be slid distally with respect to catheter body 202 to compress stent valve 230 within sheath 250.

To use catheter assembly 200, as shown in FIG. 10, using known methods, guide wire 50 is inserted into the patient's blood vessel, such as, for example, through a femoral artery, and guided toward heart 62 to the heart valve 64 that has a blockage to be cleared. After guide wire 50 is in place such that a portion of guide wire 50 extends distally of heart valve 64, catheter assembly 200 is inserted over guide wire 50 by inserting a distal end of guide wire 50 into guide wire lumen 208 in proximal end 204 of catheter body 202.

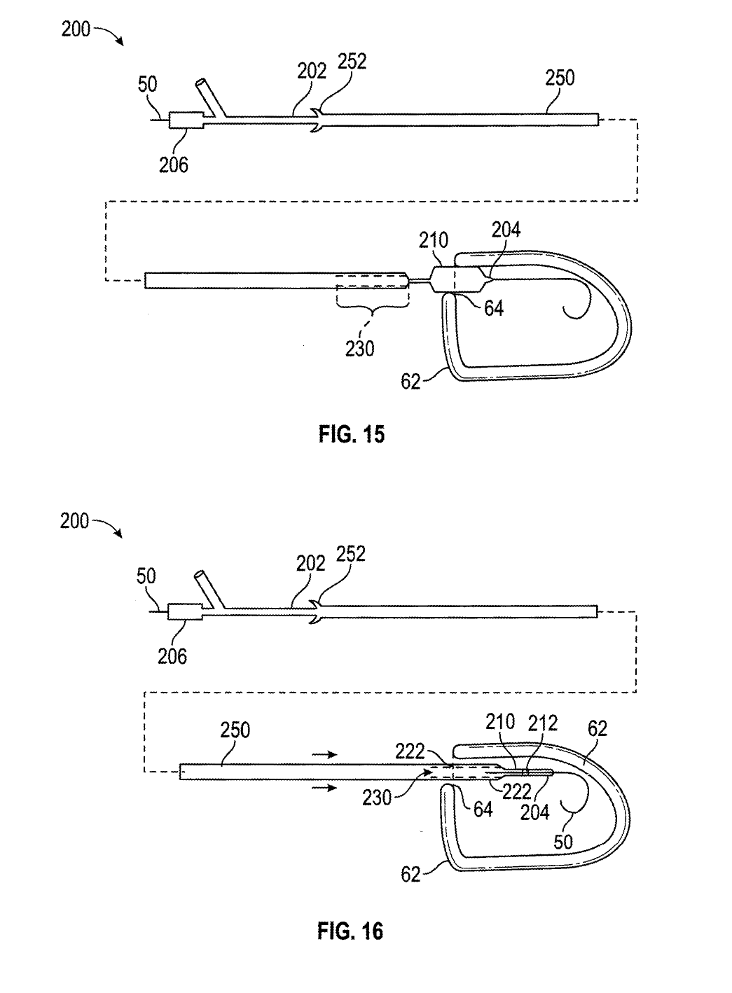

Catheter assembly 200 is then advanced distally along guide wire 50 until valvuloplasty balloon 210 is located across heart valve 64, as shown in FIG. 10. The location of valvuloplasty balloon 210 is determined by observing the location of radiopaque marker 212 within heart 62 using known techniques. Valvuloplasty balloon 210 is then inflated connecting valvuloplasty balloon inflation connection 248 to a source of inflation fluid (not shown), and pumping the inflation fluid through valvuloplasty balloon inflation connection 248 and valvuloplasty inflation lumen 249 to valvuloplasty balloon 210 to inflate valvuloplasty balloon 210 and open up heart valve 64, as shown in FIG. 15.

After valvuloplasty balloon 210 is fully expanded, and valve 64 is opened up, the fluid is released from valvuloplasty balloon 210, allowing valvuloplasty balloon 210 to contract toward its original size. Catheter assembly 200 is next advanced distally into heart 62 until stent valve 230 is located within across valve 64, as shown in FIG. 16. Stent valve 230 is advanced across valve 64 by radioscopically observing stent valve 230, which is radiopaque.

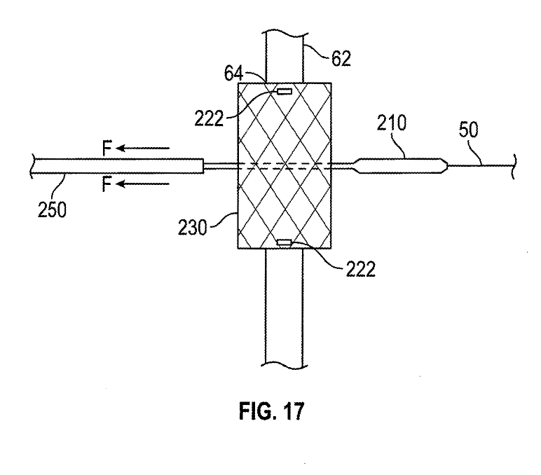

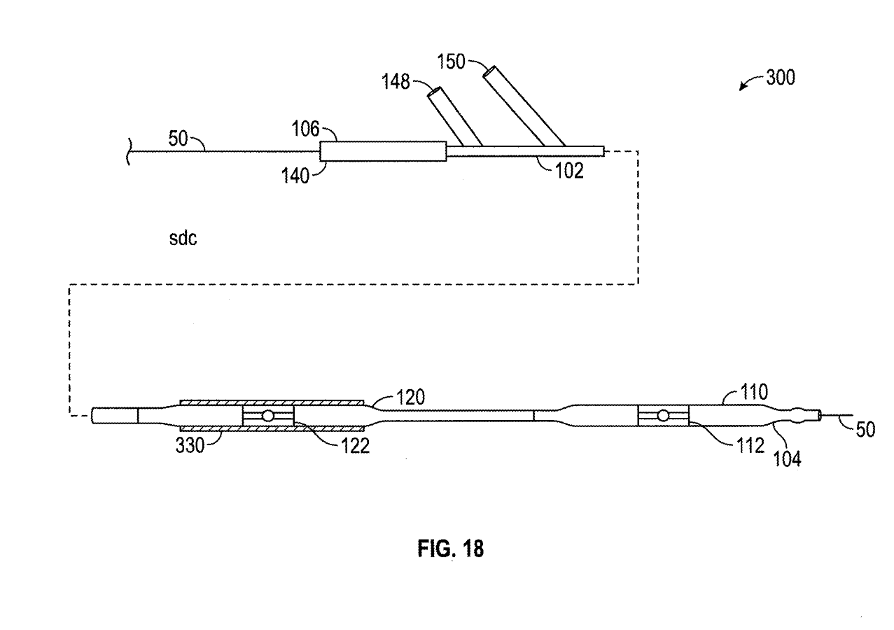

As shown in FIG. 17, valve 230 is expanded by withdrawing stent sheath 250 proximally in the direction of arrows “F”, allowing stent valve 230 to self-expand across valve 64. Next, catheter assembly 200 is withdrawn proximally through the blood vessel and removed.

Alternatively, instead of a balloon expanding stent 130, a catheter assembly 300 incorporates a balloon expandable stent valve 330. In catheter assembly 300, stent valve 330 is advanced to heart valve 64 in the same manner as described with respect to stent valve 230 above. However, instead of sliding sheath 250 proximally, stent valve balloon 120 is inflated to expand stent valve 330 across heart valve 64. In this embodiment, stent valve balloon 120 is located about 5 millimeters proximal of valvuloplasty balloon 310.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A heart valvuloplasty and stent valve deployment catheter assembly comprising:
a) a single unitary catheter body having a proximal end and a distal end, wherein the catheter body has a lumen extending between the proximal end and the distal end;
b) a guide wire extending through the lumen proximally of the proximal end and distally of the distal end;
c) a valvuloplasty balloon disposed on the body, the valvuloplasty balloon being located proximate to the distal end of the body;
d) at least one radiopaque marker disposed on the valvuloplasty balloon
e) a valvuloplasty balloon inflation connection located distally of the proximal end of the catheter body;
f) a valvuloplasty inflation lumen extending partially through the catheter body and providing fluid communication between the valvuloplasty balloon inflation connection and the valvuloplasty balloon;
g) a stent valve disposed over the body a distance of about 5 millimeters proximally of the valvuloplasty balloon; and
h) a stent valve sheath slidingly disposed over a portion of the catheter body and the stent valve.

2. The heart valvuloplasty and stent valve deployment catheter assembly according to claim 1, wherein the valvuloplasty balloon inflation connection is located proximally of the stent valve sheath.

3. The heart valvuloplasty and stent valve deployment catheter assembly according to claim 1, wherein the stent valve is a self-expanding stent valve.

4. The heart valvuloplasty and stent valve deployment catheter assembly according to claim 1, wherein the stent valve is a tricuspid one-way valve.

5. A heart valvuloplasty and stent valve deployment catheter assembly consisting of:
a) a single unitary catheter body having a proximal end and a distal end, wherein the catheter body has a lumen extending between the proximal end and the distal end;
b) a guide wire extending through the lumen proximally of the proximal end and distally of the distal end;
c) a valvuloplasty balloon disposed on the body, the valvuloplasty balloon being located proximate to the distal end of the body;
d) at least one radiopaque marker disposed on the valvuloplasty balloon
e) a valvuloplasty balloon inflation connection located distally of the proximal end of the catheter body;
f) a valvuloplasty inflation lumen extending partially through the catheter body and providing fluid communication between the valvuloplasty balloon inflation connection and the valvuloplasty balloon;
g) a stent valve disposed over the body a distance of about 5 millimeters proximally of the valvuloplasty balloon; and
h) a stent valve sheath slidingly disposed over a portion of the catheter body and the stent valve.

6. The heart valvuloplasty and stent valve deployment catheter assembly according to claim 5, wherein the valvuloplasty balloon inflation connection is located proximally of the stent valve sheath.

7. The heart valvuloplasty and stent valve deployment catheter assembly according to claim 5, wherein the stent valve is a self-expanding stent valve.

8. The heart valvuloplasty and stent valve deployment catheter assembly according to claim 5, wherein the stent valve is a tricuspid one-way valve.

9. A heart valvuloplasty and stent valve deployment catheter assembly comprising:
a) a single unitary catheter body having:
a proximal end;
a distal end;
a guide wire lumen extending between the proximal end and the distal end; and
a valvuloplasty balloon inflation lumen extending at least partially through the catheter body;
b) a valvuloplasty balloon located proximate to the distal end and in fluid communication with the valvuloplasty balloon inflation lumen; and
c) a stent valve disposed on the body, proximally of the valvuloplasty balloon.

10. The heart valvuloplasty and stent valve deployment catheter assembly according to claim 9, wherein about 5 millimeters of the catheter body extends between the valvuloplasty balloon and the stent valve.

11. The heart valvuloplasty and stent valve deployment catheter assembly according to claim 9, further comprising a valvuloplasty balloon inflation connection located proximally of the stent valve balloon and in fluid communication with the valvuloplasty balloon though the first valvuloplasty balloon inflation lumen.

12. The heart valvuloplasty and stent valve deployment catheter assembly according to claim 9, further comprising at least one radiopaque marker disposed on the valvuloplasty balloon.

13. The heart valvuloplasty and stent valve deployment catheter assembly according to claim 9, wherein the stent valve is a self-expanding stent valve.

14. The heart valvuloplasty and stent valve deployment catheter assembly according to claim 13, further comprising a stent valve sheath slidingly disposed over a portion of the catheter body and the stent valve.

15. The heart valvuloplasty and stent valve deployment catheter assembly according to claim 9, further comprising at least one radiopaque marker disposed on the stent valve.

16. The heart valvuloplasty and stent valve deployment catheter assembly according to claim 9, wherein the stent valve is a balloon expandable stent valve.

17. The heart valvuloplasty and stent valve deployment catheter assembly according to claim 9, further comprising a stent valve balloon disposed on the body at the stent valve.

18. The heart valvuloplasty and stent valve deployment catheter assembly according to claim 17, further comprising a stent valve balloon inflation connection located distally of the proximal end of the catheter body.

19. The heart valvuloplasty and stent valve deployment catheter assembly according to claim 18, further comprising a stent balloon inflation lumen extending partially through the catheter body and providing fluid communication between the stent balloon valve inflation connection and the stent valve balloon.

20. The heart valvuloplasty and stent valve deployment catheter assembly according to claim 17, wherein the stent valve balloon is located about 5 millimeters proximal of the valvuloplasty balloon.
